(19) United States
(12) Patent Application Publication
(10) Pub. No.: US 2022/0022792 A1
(43) Pub. Date: Jan. 27, 2022

(54) ELECTRODE, METHOD FOR MANUFACTURING THE SAME AND LAMINATED MATERIAL

(71) Applicant: NIPPON TELEGRAPH AND TELEPHONE CORPORATION, Tokyo (JP)

(72) Inventors: Koji SAKAI, Musashino-shi, Tokyo (JP); Tetsuhiko TESHIMA, Musashino-shi, Tokyo (JP); Yuko UENO, Musashino-shi, Tokyo (JP); Hiroshi NAKASHIMA, Musashino-shi, Tokyo (JP)

(73) Assignee: NIPPON TELEGRAPH AND TELEPHONE CORPORATION, Tokyo (JP)

(21) Appl. No.: 17/267,844

(22) PCT Filed: Aug. 7, 2019
(86) PCT No.: PCT/JP2019/031029

§ 371 Date: Feb. 11, 2021

(30) Foreign Application Priority Data
Aug. 14, 2018 (JP) 2018-152585

(51) Int. Cl. A61B 5/25

(52) U.S. Cl. CPC A61B 5/25; A61B 2562/125

(57) ABSTRACT

Provided is an electrode having an internal space, wherein the internal space is formed by a film including a layer containing a conductive material (conductive layer). Also provided is a method for producing an electrode, including a step (a) of forming a film including a layer containing a polymer compound (polymer compound layer) and a layer containing a conductive material (conductive layer); and a step (b) of allowing the film to form a tubular shape in a self-organized manner, using, s a driving force, a strain gradient in the thickness direction of the film.

### TECHNICAL FIELD

The present invention relates to an electrode, a method for producing the same, and a layered member. In particular, the present invention relates to an electrode capable of enclosing cells, a method for producing the same, and a layered member that can be used for production of the electrode.

### BACKGROUND ART

As a therapeutic method for central nervous system injury that is hard to recover, typified by spinal cord injury and cerebral infarction, expectations are rising for regenerative therapy in which living tissue is transplanted. In order to properly recover the functions of the central nervous system, it is essential to reconstruct a complex network including nerve cells as its elements, and nerve cells for compensating for lost elements and glia cells that support the activities of the nerve cells are primary cells constituting transplant tissue. For cells serving as a transplantation source, a significant development of the technology for establishing and differentiating stem cells has enabled human-derived neural cells to be produced in a diverse and selective manner. Furthermore, for the purpose of acquiring cellular activities similar to those in living bodies, and controlling the arrangement and the distribution ratio of cells, techniques for three-dimensionally folding cultured cells have been studied actively, and the techniques for designing the cell composition and the structure of transplant tissue are being established.

On the other hand, the techniques for monitoring the state of tissue after transplantation have not been sufficiently developed, and, in particular, whether a proper neural network has been reconstructed between transplant tissue and host tissue (tissue that a living body (host) in which the transplant tissue is transplanted endogenously has) is hardly revealed. As an example in which the formation of connections between nerve cells constituting transplant tissue and host tissue has been verified, a technique is available in which cells to which optical responsiveness has been imparted by an optogenetic method are transplanted in a model animal (NPL 1), but it is difficult to acquire the electrical activities for each nerve cell serving the function of the network with a sufficient space-time resolution. In addition, the technique of NPL 1 involves genetic engineering, and is therefore difficult to be applied to human host tissue.

For precise acquisition of more spatio-temporal information, electrophysiological methods in which the application of electrical stimulations and the measurement of electrical activities are performed from electrodes put in contact with cells are suitable. As compared with observation methods using imaging, the electrophysiological methods provide higher time resolution, can also obtain spatial information by using a multipoint electrode, and therefore are suitable for the purpose of evaluating the function of the network. Accordingly, for implantation of electrodes in brain and spine, various measurement devices using electrode-substrate materials having higher biocompatibility compared with that of the conventional metal electrodes have been developed intensively. In recent years, as a material for evaluation in conjunction with imaging, electrodes made of graphene, which has high light transmittance as well as biocompatibility and conductivity, are gaining attention (NPL 2). NPL 2 discloses an electrode element produced by transferring graphene to polyparaxylene (Parylene), which is a polymer material having many aromatic rings. In NPL 2, it is reported that electrical signals of living tissue are measured by implanting the above-described electrode element in living bodies.

### CITATION LIST

### Non Patent Literatures

[NPL 1] J. P. Weick et al., Human embryonic stem cell-derived neurons adopt and regulate the activity of an established neural network. Proc. Natl. Acad. Sci. USA, 2011, 108, 20189-20194

[NPL 2] D. W. Park, et al., Graphene-based carbon-layered electrodearray technology for neural imaging and optogenetic applications. Nat. Comm. 2014, 5, 5258-1-22.

### SUMMARY OF THE INVENTION

### Technical Problem

The technique described in NPL 2 has a problem in that, to be compatible with transplantation of living tissue, the technique is highly invasive to living bodies because the electrodes are implanted after tissue transplantation, and moreover, it is technically impossible to selectively bond the electrodes to the tissues, targeting each of the transplant tissue and the host tissue. In particular, if the nerve cells constituting the transplant tissue are migrating cells, it is difficult to maintain the contact between the transplant tissue and the electrodes, and therefore the process of formation of connection between the transplant tissue and the host tissue cannot be evaluated over time.

In view of the foregoing circumstances, it is an object of the present invention to provide an electrode that can enclose cells and be implanted in a living body, a method for producing the same, and a layered member for use in production of the electrode.

### Means for Solving the Problem

An aspect of the present invention is directed to an electrode having an internal space, the electrode including a film including a layer containing a conductive material (conductive layer), wherein the internal space is formed by the film being curved.

An aspect of the present invention is directed to the above-described electrode, wherein cells are present in the internal space.

An aspect of the present invention is directed to the above-described electrode, wherein the film has apertures that communicate the internal space with an external space of the electrode.

An aspect of the present invention is directed to the above-described electrode, wherein the electrode has a tubular shape.

An aspect of the present invention is directed to the above-described electrode, wherein one end or both ends of the tubular shape are closed.

An aspect of the present invention is directed to the above-described electrode, wherein the film further includes a layer containing a polymer compound (polymer compound layer).

An aspect of the present invention is directed to the above-described electrode, wherein the polymer compound layer and the conductive layer are each made of a material having light transmittance.

An aspect of the present invention is directed to the above-described electrode, wherein the conductive material is a conductive carbon material.

An aspect of the present invention is directed to a method for producing an electrode, including: a step (a) of forming a film including a layer containing a polymer compound (polymer compound layer), and a layer containing a conductive material (conductive layer); and a step (b) of allowing the film to form a three-dimensional curved shape in a self-organized manner, using, as a driving force, a strain gradient in a thickness direction of the film.

An aspect of the present invention is directed to the above-described method for producing an electrode, further including a step of (c) of allowing cells to be present on or above a surface of the film, after the step (a) and before the step (b).

An aspect of the present invention is directed to a layered member including: a substrate; a sacrificial layer stacked on the substrate; a layer containing a conductive material (conductive layer), the layer being stacked on the sacrificial layer; and a layer containing a polymer compound (polymer compound layer), the layer being stacked on the conductive layer.

### Effects of the Invention

According to the present invention, it is possible to provide an electrode that can enclose cells and be implanted in a living body, a method for producing the same, and a layered member for use in production of the electrode.

### BRIEF DESCRIPTION OF DRAWINGS

FIG. 1 shows perspective views showing an example of an electrode according to an embodiment of the present invention. FIG. 1(a) is a perspective view showing a state in which cells are enclosed in an internal space of an electrode having a tubular shape (tubular electrode). FIG. 1(b) shows a structure in which a plurality of tubular electrodes each enclosing cells (cell-enclosing tubular electrodes) are assembled. This is an example in which a three-dimensional neural network via neurites 21 is formed between the cell-enclosing electrodes. FIG. 1(c) is a schematic view showing an example in which a cell-enclosing tubular electrode has been transplanted in human brain tissue.

FIG. 2 shows schematic views illustrating an example of an electrode producing method according to an embodiment of the present invention. FIG. 2 illustrates an example of a method for producing a cell-enclosing tubular electrode.

FIG. 3 shows phase-contrast microscope images of films (graphene-Parylene electrode film) each having a layered structure in which a Parylene layer (polymer compound layer) is stacked on a graphene layer (conductive layer). Each of the films has been processed into a rectangular pattern of 600 μm long by 300 μm wide. FIG. 3(a) shows the film having no aperture, FIG. 3(b) shows the film having apertures with a diameter of 8 μm formed at intervals of 50 μm, FIG. 3(c) shows the film having apertures with a diameter of 8 μm formed at intervals of 25 μm, and FIG. 3(d) shows the film having apertures with a diameter of 15 μm formed at intervals of 50 μm.

FIG. 4 shows phase-contrast microscope images showing an enclosing process of primary culture nerve cells with self-organized curving of a graphene-Parylene electrode film. FIG. 4 shows phase-contrast microscope images obtained 0 seconds (t=0 s), 4 seconds (t=4 s), 8 seconds (t=8 s), 12 seconds (t=12 s), 16 seconds (t=16 s), or 20 seconds (t=20 s) after addition of EDTA.

FIG. 5 shows phase-contrast microscope images obtained by performing time-lapse imaging on cell-enclosing tubular electrodes in each of which cells have been enclosed by a tubular electrode formed by the graphene-Parylene electrode film. FIG. 5(a) shows the electrode having no aperture, and FIG. 5(b) shows the electrode having apertures with a diameter of 8 μm. The arrowheads in the drawing each show a representative neurite.

FIG. 6 shows a change between the electrical properties before and after self-folding of a tubular electrode. FIGS. (1) to (4) show the process for producing an electrode that is an embodiment of the present invention. FIG. 6(5) shows the results of measuring I-V curves before self-folding (dry state, in water) and after self-folding (after addition of the EDTA solution, after replacement with pure water) for the electrode that has been produced by the process shown in FIGS. 6(1) to (4).

### DESCRIPTION OF EMBODIMENTS

Hereinafter, an embodiment of the present invention will be described in detail with reference to the drawings in some cases. In the drawings, the same or corresponding constituent elements are denoted by the same reference numerals, and redundant descriptions thereof have been omitted. The proportions in the drawings may be exaggerated for the sake of description, and may not absolutely match the actual proportions.

<Electrode>

An electrode according to an embodiment of the present invention is an electrode having an internal space, the electrode including a film including a layer containing a conductive material (conductive layer), wherein the internal space is formed by the film being curved.

Hereinafter, the electrode according to this embodiment will be described with reference to the drawings showing a preferred embodiment of the present invention.

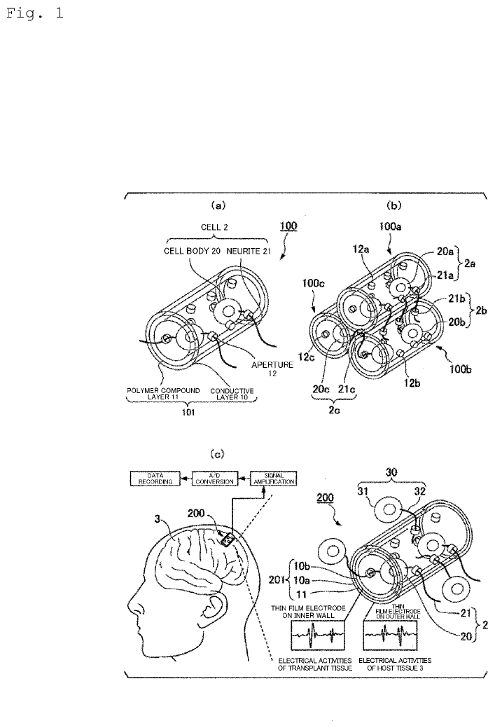

FIG. 1(a) is a perspective view showing an example of an electrode according to an embodiment of the present invention. An electrode 100 is formed by a film 101 (hereinafter referred to as an “electrode film 101”) including a layer containing a conductive material (conductive layer 10). The electrode film 101 has a three-dimensional curved shape, and an internal space of the electrode 100 is formed by the electrode film 101 being curved. In the example in FIG. 1(a), cells 2 are present in the internal space of the electrode 100, and the electrode 100 constitutes an electrode in which cells are enclosed (hereinafter also may be referred to as a “cell-enclosing electrode”).

<<Electrode>>

The electrode 100 is formed by an electrode film 101 including a conductive layer 10. The electrode film 101 has a three-dimensional curved shape, so that an internal space is formed in the electrode 100.

In the example in FIG. 1(a), the electrode film 101 includes a polymer compound layer 11, in addition to the conductive layer 10. As shown in FIG. 1(a), the electrode film 101 has a structure in which the polymer compound layer 11 is stacked on the conductive layer 10. That is to say, the conductive layer 10 is arranged on the outer side, and the polymer compound layer 11 is arranged on the inner side.

In the electrode 100 shown in FIG. 1(a), the conductive layer 10 and the polymer compound layer 11 are arranged adjacent to each other in the electrode film 101. In the electrode according to this embodiment, the conductive layer 10 and the polymer compound layer 11 do not necessarily have to be adjacent to each other, but it is preferable that the conductive layer 10 and the polymer compound layer 11 are adjacent to each other at least in a portion in which a three-dimensional curved shape is to be formed.

It is more preferable that the conductive layer 10 and the polymer compound layer 11 are in close contact with each other in a portion in which a three-dimensional curved shape is to be formed.

Although the electrode film 101 shown in FIG. 1(a) includes one conductive layer 10 and one polymer compound layer 11, the electrode film according to this embodiment is not limited to the example in FIG. 1(a). For example, as in the case of an electrode film 201 shown in FIG. 1(c), the polymer compound layer 11 may be arranged between two conductive layers, namely, a conductive layers 10a and a conductive layer 10b.

The electrode 100 has a three-dimensional curved shape. The electrode “having a three-dimensional curved shape” as mentioned herein means that at least part of the structure of the electrode has a shape that is curved in three dimensions. For example, in the example in FIG. 1(a), the electrode 100 has a shape in which the entire structure is curved in the shape of a tube (tubular shape). The tubular shape is a preferable example of the three-dimensional curved shape that the electrode 100 may have. However, the three-dimensional curved shape that the electrode 100 may have is not limited to the example in FIG. 1(a), and, for example, it is also possible that only part of the structure has a shape that is curved in three dimensions. Further examples thereof include various three-dimensional curved shapes such as a living tissue-like structure. It is possible to design the electrode 100 as those with various three-dimensional curved shapes by changing the thicknesses and the shapes of the conductive layer 10 and the polymer compound layer 11. Examples of the three-dimensional curved shape include, but are not limited to, a spherical shape and a spheroidal shape. Furthermore, the tubular shape is not limited to those having a circular cross section, and may be those having an elliptic or polygonal (triangular, quadrangular, pentagonal, hexagonal, etc.) cross section, for example. The shape of the internal space that the electrode 100 has changes according to the above-described three-dimensional curved shape, and examples thereof include a cylindrical columnar shape, a spherical shape, a spheroidal shape, a polygonal columnar shape, a polygonal pyramid shape, and a conical shape.

The size of the electrode 100 is not particularly limited, and can be selected as appropriate according to the applications of the electrode 100. For example, in the case where the cells 2 are enclosed by the electrode 100, and where the length of the cells 2 in the minor axis direction is 10 μm, the cross-sectional inner diameter of the electrode 100 is preferably larger than 10 μm, and more preferably 20 μm or more. Examples of the cross-sectional inner diameter of the electrode 100 include 20 to 200 μm, 20 to 100 μm, and 20 to 70 μm.

The size of the electrode 100 in the length direction is not particularly limited, and can be set as appropriate according to the applications of the electrode 100. For example, in the case where cells 2 are enclosed by the electrode 100, the above-described size may be a size that allows the cells 2 to be enclosed, and is preferably greater than or equal to the length of the cells 2 in the major axis direction. Examples of the size of the electrode 100 in the length direction include 20 to 10000 μm, 20 to 2000 μm, and 200 to 2000 μm.

Those practicing the invention can design the size of the electrode 100 as appropriate according to the applications of the electrode 100. In the case where the cells 2 are allowed to be present in the internal space of the electrode 100, it is possible to design the shape and the size of the electrode 100 as appropriate according to the size and the cell count of the cells 2. Furthermore, in the case where the electrode 100 in which the cells 2 are allowed to be present in the internal space is used as tissue for transplantation, it is possible to design the shape and the size of the electrode 100 as appropriate according to the purpose of the tissue for transplantation.

When the electrode 100 has a tubular shape, one or both ends of the tubular shape may be closed.

The method for closing one or both ends of the tubular shape is not particularly limited, and one or both ends of the tubular shape may be closed, for example, by plugging up any open end of the tubular shape using a suitable material. Those practicing the invention can suppress movement of the cells 2 from the internal space of the electrode 100 to the external space thereof by placing the cells 2 in the internal space of the electrode 100 and closing one or both ends of the electrode 100.

(Conductive Layer)

The conductive layer 10 is a layer containing a conductive material. There is no particular limitation on the conductive material that is used in the conductive layer 10 as long as it is conductive, but the material is preferably a nanomaterial that can be processed into a thin film shape (a material in which at least one dimension thereof is 100 nm or less). Furthermore, the material is preferably a material that does not induce a significant change in the volume when immersed in a solution, and is more preferably a material having high light transmittance and high biocompatibility. Furthermore, the conductive material is preferably a material having a n-n interaction with the polymer compound contained in the polymer compound layer 11. It is possible to increase the adhesion between the conductive layer 10 and the polymer compound layer 11, by selecting these materials.

Examples of the conductive material include conductive carbon materials such as graphene and carbon nanotube, and planar material such as molybdenum disulfide. Of these, the conductive material preferably contains a conductive carbon material, and more preferably contains graphene. The number of types of conductive materials contained in the conductive layer 10 may be one, or two or more, but is preferably one. In a preferred embodiment, the conductive layer 10 may be made of graphene, or made of buckypaper obtained by processing a carbon nanotube into a sheet form.

The conductive layer 10 may be made of a single-layered or multi-layered conductive material. In the case in which the conductive layer 10 is made of a multi-layered conductive material, there is no particular limitation on the number of layers of the conductive material, but it may be, for example, 2 to 30 layers, 2 to 20 layers, 2 to 10 layers, 2 to 5 layers, or the like. The conductive layer 10 is preferably made of a conductive carbon material with 1 to 30 layers, and the conductive layer 10 is more preferably made of a conductive carbon material with 1 to 4 layers in order to maintain the transparency of the electrode film 101. The conductive layer 10 is even more preferably made of graphene with 1 to 30 layers, and the conductive layer 10 is particularly preferably made of graphene with 1 to 4 layers in order to maintain the transparency of the electrode film 101. In the case where the conductive layer 10 is made of graphene, it may be either polycrystalline graphene or single crystal graphene, but it is preferably single crystal graphene from the viewpoint of controlling the direction of the curved shape.

Of these, the conductive material is preferably graphene. Graphene has high biocompatibility, and thus is less likely to cause post-implant inflammation in the case where the electrode 100 is implanted in a living body. Furthermore, due to the high transparency, it also enables evaluation in conjunction with imaging. Graphene has a light transmittance of 97.7%, and has higher light transmittance as compared with conductive metal materials such as gold, silver, and copper.

The thickness of the conductive layer 10 is preferably 0.3 to 10 nm. If the conductive layer 10 is made of a multi-layered conductive material, the total thickness of the plurality of layers is the thickness of the conductive layer 10. Those practicing the invention can allow the electrode film 101 to form a three-dimensional curved shape in a self-organized manner, by setting the thickness of the conductive layer 10 to the above-described range and setting the thickness of the polymer compound layer 11 to a later-described predetermined range. From the viewpoint of forming a stable three-dimensional curved shape, the thickness of the conductive layer 10 is preferably 0.3 to 7 nm, more preferably 0.3 to 5 nm, and even more preferably 0.3 to 1.2 nm. It is possible to obtain an electrode 100 having any three-dimensional curved shape, by controlling the ratio of the thickness of the conductive layer 10 with respect to the thickness of the polymer compound layer 11 within the above-described range. For example, it is possible to reduce the radius of curvature of the three-dimensional curved shape, by increasing the thickness of the conductive layer 10 with respect to the thickness of the polymer compound layer 11.

If the electrode film has a configuration in which the polymer compound layer 11 is arranged between the two conductive layers 10a and 10b as in the case of the electrode film 201 shown in FIG. 1(c), the thickness of the conductive layer 10a on the outer side is preferably 0.3 to 7 nm, and more preferably 0.3 to 1.2 nm. The thickness of the conductive layer 10b on the inner side is preferably 0.3 to 7 nm, and more preferably 0.3 to 1.2 nm.

The thickness ratio between the conductive layer 10a on the outer side and the conductive layer 10b on the inner side (the thickness of the conductive layer 10a/the thickness of the conductive layer 10b) is preferably in the range of 1 to 10, and more preferably in the range of 2 to 4.

(Polymer Compound Layer)

The electrode film 101 preferably includes a polymer compound layer 11, in addition to the above-described conductive layer 10. The polymer compound layer 11 is preferably a layer containing a polymer compound having an aromatic ring. The polymer compound that is used in the polymer compound layer 11 is preferably a compound having many aromatic rings in molecules, and having a n-n interaction with the conductive material contained in the conductive layer 10. With such a polymer compound, the adhesion of the polymer compound layer 11 to the conductive layer 10 increases.

Furthermore, the polymer compound layer 11 is preferably made of a material having high light transmittance and high biocompatibility, and it is more preferable to use a polymer compound that is not toxic to cells. Examples of such a polymer compound include polyparaxylene and a derivative thereof. Examples of the derivative of polyparaxylene include polymers such as halogenated paraxylene (chloroparaxylene, fluoroparaxylene, etc.).

Of these, the polymer compound is preferably polyparaxylene. Polyparaxylene has high biocompatibility, and thus is less likely to cause post-implant inflammation in the case where the electrode 100 is implanted in a living body. Furthermore, due to the high transparency, it also enables evaluation in conjunction with imaging. Furthermore, since a thin film of polyparaxylene is flexible and durable, it is possible to maintain the three-dimensional curved structure of the electrode 100 even with a nanometer-level thin film.

In addition, polyparaxylene has highly insulating, and thus can prevent conduction at the conductive layer 10a and the conductive layer 10b in the case where the polymer compound layer 11 is arranged between the two conductive layers 10a and 10b as shown in FIG. 1(c). Accordingly, those practicing the invention can selectively measure the electrical activities of each of the cells 2 enclosed by the electrode 200 and host tissue 3 and selectively applying an electrical stimulation to each of the cells 2 and the host tissue 3 in FIG. 1(c).

Polyparaxylene has high adhesion to graphene, and thus can be particularly preferably used in the case where graphene is used as the conductive material of the conductive layer 10. Those practicing the invention can make peeling and breakage less likely to occur by using graphene as the conductive layer 10 and using polyparaxylene as the polymer compound layer 11, thus making it possible to form a desired three-dimensional curved shape without loss of conductivity.

The number of types of polymer compounds contained in the polymer compound layer 11 may be one, or two or more, but is preferably one.

The thickness of the polymer compound layer 11 is preferably 10 to 900 nm. If the polymer compound layer 11 is made of a multi-layered thin film, the total thickness of the plurality of layers of the thin film is the thickness of the polymer compound layer 11. Those practicing the invention can allow the electrode film 101 to form a three-dimensional curved shape in a self-organized manner, by setting the thickness of the polymer compound layer 11 to the above-described range and setting the thickness of the conductive layer 10 to the above-described predetermined range. From the viewpoint of forming a stable three-dimensional curved shape, the thickness of the polymer compound layer 11 is preferably 40 to 400 nm, and more preferably 50 to 250 nm. Those practicing the invention can obtain an electrode 100 having any three-dimensional curved shape, by controlling the ratio of the thickness of the polymer compound layer 11 with respect to the thickness of the conductive layer 10 within the above-described range. For example, those practicing the invention can increase the radius of curvature of the three-dimensional curved shape, by increasing the thickness of the polymer compound layer 11 with respect to the thickness of the conductive layer 10.

There is no particular limitation on the thickness ratio between the conductive layer 10 and the polymer compound layer 11 (the thickness of the conductive layer 10/the thickness of the polymer compound layer 11) as long as it is from 1/3000 to 1/1, but it is preferably from 1/1200 to 1/4. Those practicing the invention can form a stable three-dimensional curved shape, by setting the thickness ratio between the conductive layer 10 and the polymer compound layer 11 to the above-described range.

If the electrode film has a configuration in which the polymer compound layer 11 is arranged between the two conductive layers 10a and 10b as in the case of the electrode film 201 shown in FIG. 1(c), the thickness of the polymer compound layer 11 is preferably 40 to 400 nm, and more preferably 50 to 250 nm.

The thickness ratio between the conductive layer 10a on the outer side and the polymer compound layer 11 (the thickness of the conductive layer 10a/the thickness of the polymer compound layer 11) is preferably in the range of 1/3000 to 1/1, and more preferably in the range of 1/1200 to 1/4. The thickness ratio between the conductive layer 10b on the inner side and the polymer compound layer 11 (the thickness of the conductive layer 10b/the thickness of the polymer compound layer 11) is preferably in the range of 1/3000 to 1/1, and more preferably in the range of 1/1200 to 1/4.

(Apertures)

The electrode film 101 may have apertures 12 that communicate the internal space of the electrode 100 with the external space of the electrode 100. If the electrode film 101 has apertures 12, the apertures 12 are apertures extending through the electrode film 101. By having the apertures 12, the electrode film 101 allows the exchange of substances between the internal space and the external space of the electrode 100. Furthermore, if cells 2 are enclosed in the internal space of the electrode 100, the cells 2 can access the external space via the apertures 12. Here, accessing the external space is meant by the content described below. The content refers to that cells that are present in the internal space of the electrode 100 act on or are affected by the external space (external environment) of the electrode via the above-described apertures. Specific examples of cases where the cells 2 access the external space of the electrode 100 will be given below: The examples include that, via the apertures 12, the cells 2 incorporate a substance from the external space of the electrode 100, the cells 2 discharge a substance to the external space, the cells 2 come into contact with a substance in the external space, the cells 2 interact with cells in the external space, and the cells 2 each extend its part such as a neurite into the external space.

The electrode 100 shown in FIG. 1(a) has apertures 12 extending through the conductive layer 10 and the polymer compound layer 11. The cells 2 enclosed by the electrode 100 each include a cell body 20 and a neurite 21, and the neurites 21 pass through the apertures 12 and extend to the outside of the electrode 100.

The shape of the apertures 12 is not particularly limited, and can be any shape. Examples of the cross-sectional shape of the apertures 12 include, but are not limited to, a circular shape, an elliptic shape, and a polygonal shape (a triangular shape, a quadrangular shape, a hexagonal shape, etc.). In view of the ease of formation, for example, the apertures 12 preferably have a circular shape or an elliptic shape.

The inner diameter of the apertures 12 is not particularly limited, and can be set as appropriate according to the purpose. The inner diameter of the apertures 12 is preferably larger than the size of an object that is to be passed through the apertures 12, and is preferably smaller than the size of an object that is not to be passed through the apertures 12. For example, in the case where the electrode film 101 encloses the cells 2, the inner diameter of the apertures 12 is preferably smaller than the length of each cell 2 in the minor axis direction. For example, if the length of the cell body 20 of each cell 2 in the minor axis direction is about 10 μm, the inner diameter of the apertures 12 is preferably less than 10 μm. Furthermore, in order to allow a neurite 21 with a diameter of 0.1 to 2 μm to pass through the apertures 12, the inner diameter of the apertures 12 is preferably 1 μm or more, and more preferably 3 μm or more. Examples of the inner diameter of the apertures 12 include 1 to 15 μm, 1 to 10 μm, and 3 to 8 μm.

The shape or the size of the apertures 12 may be changed in the thickness direction of the electrode film 101. For example, the apertures 12 may have a conical shape. For example, those practicing the invention can induce the extension of the neurite 21 to the external space of the electrode 100, by forming the apertures 12 into a shape that is tapered from the internal space of the electrode 100 toward the external space thereof.

For example, if the apertures 12 are holes that are recessed outward from the inner side of the electrode 100 (the inward inner diameter>the outward inner diameter), the neurite 21 is likely to extend from the inner side to the outer side. Furthermore, those practicing the invention can control the ratio of the number of neurites 21 passing through the apertures 12, by adjusting the size of the apertures 12. The ratio of the excitation propagations between the nervous tissues inside and outside the electrode 100 can be adjusted through the balance between the numbers of neurites 21 extending to the inside and the outside of the electrode 100.

There is no particular limitation on the arrangement of the apertures 12, as long as it allows the electrode 100 to maintain the three-dimensional curved shape. The arrangement of the apertures 12 may be in a grid configuration, or may be in a zigzag configuration. From the viewpoint of preferably maintaining the three-dimensional curved shape of the electrode 100, examples of the interval between the apertures 12 include intervals at which the center-to-center distance between adjacent apertures 12 is about 25 to 500 μm. Note that the apertures 12 may be formed such that the electrode 100 is in a mesh form, as long as the three-dimensional curved shape of the electrode 100 can be maintained.

In the case where the electrode 100 encloses the cells 2, the electrode 100, by having the apertures 12, allows each of the cells 2 to extend a neurite 21 or the like to the external environment via the apertures 12. Furthermore, the cells enclosed by the electrode 100 can discharge a substance such as nitrogen monoxide and potassium to the external environment of the electrode 100, or can incorporate a substance such as oxygen and sugar from the external environment, via the apertures 12. Accordingly, those practicing the invention can culture the cells 2 for a long period of time in a state in which the cells 2 are enclosed by the electrode 100.

<<Cell>>

Preferably, in the electrode 100, cells 2 are present in the internal space. That is to say, the electrode 100 is preferably a cell-enclosing electrode. Those practicing the invention can use the cell-enclosing electrode as transplant tissue to a living body, and can monitor the electrical activities of the transplant tissue and host tissue using this electrode.

The electrode 100 depicted in FIG. 1(a) encloses the cells 2. In the example in FIG. 1(a), the cells 2 are nerve cells each including a cell body 20 and neurites 21. The neurites 21 may each be either a dendrite or an axis cylinder of a nerve cell. Although the cells 2 are nerve cells in the example in FIG. 1(a), the cells 2 are not limited to nerve cells, and may be other types of cells.

Each cell 2 may be an animal cell, or may be a plant cell, but is preferably an animal cell. The animal cell is preferably a mammalian cell. Examples of the mammalian cell include human cells, cells of mammals other than human. Examples of the cells of mammals other than human include cells of primates (chimpanzee, gorilla, monkey, etc.), cells of livestock animals (cow, pig, sheep, horse, etc.), cells of rodents (mouse, rat, guinea pig, hamster, etc.), and cells of pets (dog, cat, rabbit, etc.).

There is no particular limitation on the cell type of the cells 2, and the cells 2 may be any cells in a living body. Examples thereof include nerve cells, glia cells, myocardial cells, fibroblasts, and vessel epithelial cells.

In the case where the electrode 100 of this embodiment is used as nervous tissue for transplantation by enclosing cells, preferable examples of the cells 2 include nerve cells and glia cells. The cells 2 may be one type of cells, or may be a mixture of a plurality of types of cells. Preferable examples of the mixture of cells include a mixture of nerve cells and glia cells.

The number of cells 2 enclosed in the internal space of the three-dimensional curved shape of the electrode 100 is not particularly limited, and may be any number corresponding to the size of the internal space of the three-dimensional curved shape of the electrode 100. The cells 2 can be cultured while being enclosed by the electrode 100, and may proliferate inside the internal space of the electrode 100. Accordingly, regardless of the number of cells 2 that are initially enclosed, the internal space of the electrode 100 can be filled with a proper number of cells 2, by continuing culturing.

The cells 2 are present in the internal space of the electrode 100. “Cells are present in the internal space of the electrode” as mentioned herein means that at least part of each cell is present in the internal space of the three-dimensional curved shape formed by the electrode film, and the whole cell does not need to be present in the internal space.

For example, a state in which the cell body 20 is present in the internal space of the electrode 100, and the neurite 21 extends to the external environment of the electrode 100 as shown in FIG. 1(a) is also encompassed by the state “cells are present in the internal space of the electrode”.

(Other Constituent Elements)

The electrode 100 may have other constituent elements in addition to the conductive layer 10 and the polymer compound layer 11 described above, within the range not impairing the effects of the present invention. Examples of the other constituent elements include a protein layer and a metal layer.

Protein Layer

The electrode 100 may include a protein layer. The protein layer is a layer containing protein as a main component. The protein layer maybe arranged, for example, on one or both of the innermost layer and the outermost layer in the electrode 100.

Examples of the protein that forms the protein layer include, but are not limited to, extracellular matrices such as fibronectin, collagen, and laminin, and may be selected as appropriated according to the applications of the electrode 100, the type of the cells 2, and the host tissue in which the cells 2 are to be transplanted. Any function can be imparted to the electrode 100 by providing the protein layer. For example, in the case where an extracellular matrix as described above is used as the protein, it is possible to increase the adhesion between the electrode 100 with the cells 2 or the cells of the host tissue.

Metal Layer

The electrode 100 may include a metal layer. The metal layer is a layer containing a metal element. When evaluating the electrical properties of the electrode 100, in particular when allowing the current to flow at an end thereof using a probe, if a single-layered graphene film or the like is used as the conductive layer 10, it is difficult to directly putting an end of the probe into close contact with the conductive layer 10. If the electrode 100 has a metal layer, a mechanical strength that can prevent the layer from coming off due to the probe can be imparted to the electrode 100, and the shape of the electrode can be maintained. There is no particular limitation on the metal element that is contained in the metal layer as long as it is an element that is commonly used in metal electrodes, but examples thereof include noble metals such as gold, silver, platinum, palladium, rhodium, iridium, ruthenium, and iridium. There is no particular limitation on the thickness of the metal layer, but it is preferably, for example, 10 nm to 100 μm. If the electrode 100 includes a metal layer, the metal layer is preferably arranged at a portion not having a three-dimensional curved shape.

In the electrode 100, the thickness of the entire electrode film 101 of a portion having a three-dimensional curved shape (the total thickness of the conductive layer 10 and the polymer compound layer 11) is preferably approximately 10 to 500 nm, in order not to hinder bending in a later-described production step.

In a preferable specific examples of the electrode 100, the conductive layer 10 is made of graphene, the polymer compound layer 11 is made of polyparaxylene or a derivative thereof, and the conductive layer 10 and the polymer compound layer 11 are adjacent to each other. Since graphene and polyparaxylene or a derivative thereof have particularly high adhesion to each other, this configuration can suppress occurrence of coming off, slippage, breakage, and the like even in a three-dimensional curved shape portion. Preferable specific examples of the electrode 100 include an electrode in which cells 2 are present in the internal space. In particular, the cells 2 are preferably nerve cells, glia cells, or a mixture of nerve cells and glia cells. Furthermore, the electrode film 101 preferably has apertures 12.

<<Usage Example>>

FIG. 1(b) shows a structure in which a plurality of tubular electrodes 100a to 100c are assembled. Cells 2a to 2c, which are nerve cells, are present in the internal spaces of the electrodes 100a to 100c, respectively. Those practicing the invention can allow a three-dimensional network to be formed between the cells 2a to 2c respectively enclosed by the electrodes 100a to 100c, by assembling the plurality of electrodes 100a to 100c. In FIG. 1(b), neurites 21a to 21c extend from apertures 12a to 12c of the electrodes 100a to 100c and are connected to each other, so that a three-dimensional neural network is formed.

FIG. 1(c) is a schematic view showing an example in which a cell-enclosing electrode is transplanted in human brain tissue. The cell-enclosing electrode 200 shown in FIG. 1(c) has a configuration in which the cells 2 are enclosed by the electrode film 201 including the two conductive layers 10a and 10b and the polymer compound layer 11 sandwiched therebetween (see the right part in FIG. 1(c)). Each of the cells 2 enclosed by the cell-enclosing electrode 200 that has been transplanted to human brain tissue (host tissue 3) extends its neurite 21 to the host tissue 3 via an aperture 12 formed in the electrode film 201. Each nerve cell 30 in the host tissue 3 also extends its neurite 32 to the internal space of the cell-enclosing electrode 200 via an aperture 12. Then, a synaptic connection is formed between the cell 2 and the nerve cell 30 of the host tissue 3 via the neurites 21 and 32. Those practicing the invention can measure the electrical activity of the host tissue 3 by the conductive layer 10a on the outer side, and can measure the electrical activities of the cells 2 enclosed by the cell-enclosing electrode 200 by the conductive layer 10b on the inner side. Electrical signals that have been respectively measured by the conductive layer 10a and the conductive layer 10b may be amplified and A/D converted, and the resulting measured data may be recorded by an external recording device.

With the electrode according to this embodiment, cells constituting transplant tissue are enclosed by the electrode, so that it is possible to allow both measurement of the electrical activities of the transplant tissue and electrical stimulation to the transplant tissue in the case where the above-described cell-enclosing electrode is transplanted in a living body. Furthermore, those practicing the invention can put the electrode into contact with each of the transplant tissue and the host tissue, by forming the electrode film in a configuration in which a polymer compound layer is sandwiched between two conductive layers, and can selectively perform each of the measurement of the electrical activities of the transplant tissue and the host tissue and the electrical stimulation to the transplant tissue and the host tissue.

With the electrode according to this embodiment, it is possible to inhibit loss of cell-electrode contacts due to cell migration, by enclosing transplant cells in the internal space of the electrode. Therefore, the activities of the transplant cells can be stably measured over a long period of time. Accordingly, it is possible to monitor, over a long period of time, a process in which the transplant tissue is connected to the host tissue and restores the host tissue, and evaluate the process.

Furthermore, it is not necessary to separately implant the transplant tissue and the electrode, and therefore the implanting operation needs to be performed only once, resulting in a reduction in invasiveness. Unlike imaging techniques that involve genetic engineering, such as an optogenetic method, the electrode does not require genetic engineering, and is also applicable to human.

With the electrode according to this embodiment, the substance exchange in a liquid present in the internal space of the electrode is reduced. In the case where the extracellular potential change caused by the electrical activities of nerve cells is measured by the electrode, the leakage of ion current can be prevented under an environment in which a substance is less likely to diffuse, and it is thus possible to obtain a signal with a large amplitude. In addition, with the electrode according to this embodiment, the electrode can be transplanted to the host tissue in a state in which cells are enclosed in the internal space, and therefore the state of contact between the electrode and the cells is favorable. Accordingly, with the electrode of this embodiment, it is possible to obtain a signal with a higher S/N ratio as compared with a case where a typical insertable electrode is used.

The electrode according to this embodiment can also be used as a scaffold for folding a three-dimensional tissue. In order to construct a functional transplant tissue, a scaffold for folding a three-dimensional tissue made of many types of neural cells is required. The electrode of this embodiment can be operated with a micromanipulator, and therefore those practicing the invention can construct transplant tissues in various designs by assembling (folding) a plurality of cell-enclosing electrodes. In particular, those practicing the invention can combine a plurality of cell-enclosing electrodes to form a three-dimensional transplant tissue having a neural network with any structure, by using a cell-enclosing electrode having apertures as the cell-enclosing electrode according to this embodiment.

If the cell-enclosing electrode according to this embodiment has apertures, the cells enclosed in the electrode can access the external environment via the apertures. For example, via the apertures, the extension of neurites, the migration of glia cells, and the substance exchange between the internal environment and the external environment of the electrode can be allowed, and the migration of nerve cells can be prevented by the wall surface of the electrode. Accordingly, if the enclosed cells are nerve cells, for example, the neurites can be extended to the external environment via the apertures while retaining the nerve cells in the internal space of the electrode. Furthermore, the nerve cells in the external environment can also extend neurites to the internal space of the electrode via the apertures. Accordingly, it is possible to realize synapse formation in tissues inside and outside the electrode. Furthermore, it is possible to inhibit the necrosis or the like of the cells enclosed by the electrode.

Those practicing the invention can control conditions such as the migration of the cells enclosed by the cell-enclosing electrode, the extension of neurites, and the substance exchange (the release and the incorporation of substances) inside and outside the electrode, by controlling the size of the apertures present in the electrode film. Therefore, the electrode of this embodiment is not only applicable as transplant tissue, but also applicable as an experimental system for evaluating the effects of transplantation. Although the effects of cell transplantation to a living body can be broadly classified into cytokine released by transplant cells, and the interaction via contact between the transplant cells and the cells of host tissue, the magnitude of the influence of each of these effects is hardly known. By varying the size of the apertures in the cell-enclosing electrode of this embodiment, those practicing the invention can apply the cell-enclosing electrode as an experimental system for evaluating the influence of the transplant cells on a living body.

If the conductive layer is made of graphene and the polymer compound layer is made of polyparaxylene in the electrode of this embodiment, the electrode is less likely to cause an inflammatory response even when implanted in a living body since graphene and polyparaxylene are materials having high biocompatibility. A conventional metal electrode is problematic in that if the electrode is implanted in a living body, glial scar formed around the electrode due to an inflammatory response prevents a contact between the electrode and the cells, resulting in loss of signals measured by the electrode. With the electrode of this embodiment, it is possible to inhibit the inflammatory response after the electrode has been implanted in a living body, by using, in particular, graphene and polyparaxylene, thus maintaining the contact between the electrode and the cells. Accordingly, even after the electrode has been implanted in a living body, it is possible to measure the electrical activities of the transplant cells for a long period of time.

<Electrode Producing Method>

A method for producing an electrode according to an embodiment of the present invention includes: a step (a) of forming a film including a layer containing a polymer compound (polymer compound layer), and a layer containing a conductive material (conductive layer); and a step (b) of allowing the film to form a three-dimensional curved shape in a self-organized manner, using, as a driving force, a strain gradient in a thickness direction of the film. Preferably, the electrode producing method of this embodiment further includes a step of (c) of allowing cells to be present on or above a surface of the film, after the step (a) and before the step (b). Hereinafter, the electrode producing method of the present invention will be described with reference to the drawings showing a preferred embodiment of the present invention.

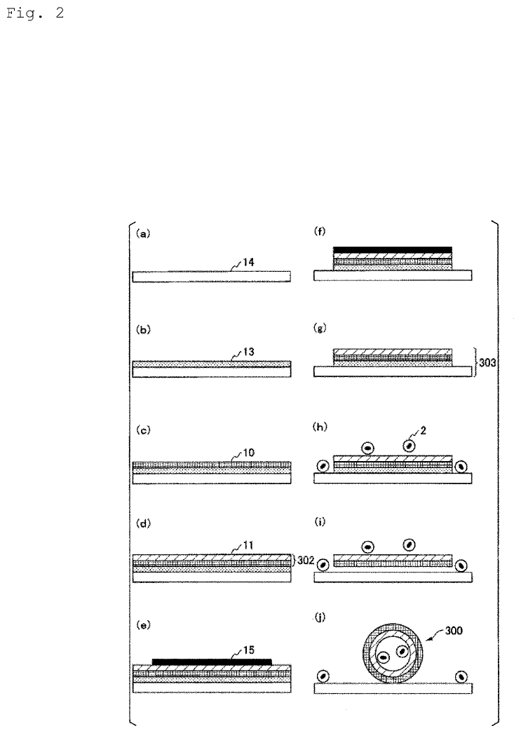

FIG. 2 shows views showing the outline of the electrode producing method according to an embodiment of the present invention.

First, a film 302 including the conductive layer 10 and the polymer compound layer 11 (hereinafter referred to as an “electrode film 302”) is formed (FIGS. 2(a) to 2(g) : step (a)). In the example in FIGS. 2(a) to 2(g), a sacrificial layer 13 is formed on a substrate 14, and the conductive layer 10 and the polymer compound layer 11 are formed on the sacrificial layer 13, so that the electrode film 302 is formed.

Then, cells 2 are allowed to be present on or above the surface of the electrode film 302 (FIG. 2(h): step (c)).

Then, the electrode film 302 is allowed to form a three-dimensional curved shape in a self-organized manner, using, as a driving force, a strain gradient in the thickness direction of the electrode film 302 (FIGS. 2(i) to 2(j): step (b)). In the example in FIGS. 2(i) to 2(j), the conductive layer 10 and the polymer compound layer 11 are put into close contact with and connected to each other, so that stress distribution appears in the thickness direction of the electrode film 302, and a strain gradient in the in-plane direction of the electrode film 302 is formed by separating the electrode film 302 from the substrate 14 by decomposing the sacrificial layer 13 (FIG. 2(i)). Using this strain gradient as a driving force, the conductive layer 10 and the polymer compound layer 11 are bent while being in close contact with each other (FIG. 2(i)), and thus a three-dimensional curved shape is obtained in a self-organized manner (FIG. 2(j)). When being bent, the electrode film 302 forms a three-dimensional curved shape while enclosing the cells 2 present on or above the surface, and it is thus possible to obtain an electrode 300 in which the cells 2 are enclosed in the internal space of the three-dimensional curved shape.

Hereinafter, the steps of the electrode producing method according to this embodiment will be described.

[Step (a)]

The step (a) is a step of forming a film including a polymer compound layer and a conductive layer (layered member).

Examples of the method for forming the film including a polymer compound layer and a conductive layer include, but are not particularly limited to, a method that uses a substrate and a sacrificial layer. In the example in FIGS. 2(a) to 2(g), a sacrificial layer 13 is formed on a substrate 14 (FIGS. 2(a) to 2(b)), then a conductive layer 10 is formed on the sacrificial layer 13 (FIG. 2(c)), and then a polymer compound layer 11 is formed on the conductive layer 10 (FIG. 2(d)), so that the electrode film 302 is formed. By forming the electrode film 302 on the substrate 14 and the sacrificial layer 13, the electrode film 302 can be formed while maintaining the two-dimensional plane structure.

(Substrate)

The substrate 14 is used for the sake of convenience for formation of the electrode film 302, and there is no particular limitation on its material. The material of the substrate 14 is preferably a material with high surface flatness. Furthermore, if observation is performed using a fluorescence microscope or the like in a state in which a cell is enclosed by the electrode produced according to the method of this embodiment that has been held on the substrate, it is preferably a material that does not disturb the observation of the fluorescence intensity of the cell using the fluorescence microscope and that has a wavelength absorption band as optical properties not overlapping that of the conductive layer 10.

Examples of the material of the substrate 14 include silicon, soda glass, quartz, magnesium oxide, sapphire, and the like.

There is no particular limitation on the thickness of the substrate 14, but it is preferably approximately 50 to 200 μm.

Specific examples of the substrate 14 include a glass substrate with a thickness of approximately 100 μm.

(Sacrificial Layer)

The sacrificial layer 13 has a function as a temporary adhesion layer for separating the electrode film 302 including the conductive layer 10 and the polymer compound layer 11 from the substrate 14. There is no particular limitation on the material for forming the sacrificial layer 13 as long as it is a material that melts in response to a stimulation from the outside such as a chemical material, a change in the temperature, irradiation with light, or the like. Examples of the sacrificial layer 13 include a calcium alginate gel, which is a type of physical gel. The calcium alginate gel melts through transition of the calcium alginate gel from gel to sol in response to the addition of a chelating agent such as sodium citrate or ethylenediaminetetraacetic acid (EDTA), an enzyme called arginase, or the like. Since the sacrificial layer 13 melts in response to a stimulation from the outside, it is possible to separate the electrode film 302 from the substrate 14 by melting the sacrificial layer 13 in a later-described step (c), thereby allowing the electrode film 302 to form a three-dimensional curved shape in a self-organized manner.

Since the chelating agent such as sodium citrate or ethylenediaminetetraacetic acid (EDTA) is not toxic to biological samples such as cell, a target cell can be enclosed by suspending the cell immediately before melting of the sacrificial layer 13.

The material of the sacrificial layer 13 maybe any other materials regardless of the type such as synthetic polymers or biopolymers, as long as they are materials that melt in response to a stimulation from the outside. Preferred examples thereof include a thin metal film that can be melted by an etchant, poly(N-isopropylacrylamide) whose gel-sol transition can be induced by a change in the temperature, photoresists whose gel-sol transition can be induced by irradiation with ultraviolet light, and the like.

There is no particular limitation on the thickness of the sacrificial layer 13. The thickness of the sacrificial layer 13 maybe, for example, 20 to 1000 nm, from the viewpoint of realizing quick melting.

There is no particular limitation on the method for forming the sacrificial layer 13 on the substrate 14, and methods commonly used to form a thin film can be selected as appropriate according to the material of the sacrificial layer 13. Examples of the method for forming the sacrificial layer 13 include chemical vapor deposition (CVD), spin coating, inkjet printing, vapor deposition, and electrospraying.

(Conductive Layer, Polymer Compound Layer)

The conductive layer 10 and the polymer compound layer 11 are the same as those described in the section “<Electrode>” above.

In the example in FIGS. 2(c) and 2(d), the conductive layer 10 is formed on the sacrificial layer 13, and then the polymer compound layer 11 is formed on the conductive layer 10, but the forming order maybe opposite. That is to say, it is also possible that the polymer compound layer 11 is formed on the sacrificial layer 13, and then the conductive layer 10 is formed on the polymer compound layer 11.

The thickness of the conductive layer 10 formed at this time is preferably 0.3 to 10 nm, preferably 0.3 to 7 nm, and more preferably 0.3 to 1.2 nm. The thickness of the polymer compound layer 11 is preferably 10 to 900 nm, preferably 40 to 400 nm, and more preferably 50 to 250 nm. It is possible to form a strain gradient in the thickness direction of the electrode film 302 by setting the thicknesses of the conductive layer 10 and the polymer compound layer 11 within the above-described range. If the thickness of the conductive layer 10 is increased within the above-described range, the radius of curvature of the three-dimensional curved shape formed in a later-described step (c) can be reduced. On the other hand, if the thickness of the polymer compound layer 11 is increased within the above-described range, the radius of curvature of the three-dimensional curved shape formed in a later-described step (c) can be increased.

There is no particular limitation on the method for forming the conductive layer 10, and examples thereof include transfer using a water surface, chemical vapor deposition (CVD), spin coating, inkjet printing, thermal vapor deposition, and electrospraying. For example, if the conductive layer 10 is made of graphene, it is possible to form the conductive layer 10 by forming a single-layered graphene film through CVD on the surface of a metal film such as a copper foil, melting the metal film, repeating washing on the water surface, and thereafter transferring the single-layered graphene film to the surface of the sacrificial layer 13 or the polymer compound layer 11. Moreover, it is possible to form the conductive layer 10 made of a multi-layered graphene by repeating the above-described processing.

There is no particular limitation on the method for forming the polymer compound layer 11, and it i s possible to use CVD, spin coating, inkjet printing, vapor deposition, electrospraying, and the like. For example, if the polymer compound layer 11 is made of polyparaxylene or a derivative thereof, it is possible to form the polymer compound layer 11 by causing a dimer of the paraxylene or derivative thereof to grow through CVD.

If the electrode has a configuration in which the polymer compound layer 11 is sandwiched between the two conductive layers 10a and 10b as in the case of the cell-enclosing electrode 200 shown in FIG. 1(c), the conductive layer 109a may be formed on the sacrificial layer 13, then polymer compound layer 11 is formed on the conductive layer 10a, and thereafter the conductive layer 10b may be formed on the polymer compound layer 11. In this case, the thickness of the conductive layer 10a may be 0.3 to 10 nm, preferably 0.3 to 7 nm, and more preferably 0.3 to 1.2 nm. The thickness of the polymer compound layer 11 may be 10 to 900 nm, preferably 40 to 400 nm, and more preferably 50 to 250 nm. The thickness of the conductive layer 10b may be 0.3 to 10 nm, preferably 0.3 to 7 nm, and more preferably 0.3 to 1.2 nm.

(Other Constituent Elements)

The electrode film 302 formed in this step may include other constituent elements in addition to the conductive layer 10 and the polymer compound layer 11 described above. There is no particular limitation on the other constituent elements, and they can be selected as appropriate according to the purpose. Examples of the other constituent elements included in the electrode film 302 include an additional layer other than the conductive layer 10 and the polymer compound layer 11. The thickness, the forming method, and the like of the additional layer can be selected as appropriate according to the material for forming the layer. Examples of the additional layer include a protein layer and a metal layer.

If the electrode film 302 includes a protein layer, the protein layer is preferably formed on the uppermost layer of the electrode film 302. That is to say, in the example in FIG. 2, the protein layer is preferably formed on the polymer compound layer 11. Examples of the protein that forms the protein layer are the same as those given in the section “<Electrode>” above.

Examples of the method for forming the protein layer include, but are not particularly limited to, a method in which the electrode film 302 is immersed in a protein solution or a protein suspension.

If the electrode film 302 includes a metal layer, the thickness of the metal layer may be 10 nm to 100 μm, for example. The metal layer may be provided adjacent to the conductive layer 10, and it is possible that, after the conductive layer 10 is formed on the sacrificial layer 13, the metal layer is formed on the conductive layer 10. Alternatively, it is also possible that, after the polymer compound layer 11 is formed on the sacrificial layer 13, the metal layer is formed on the polymer compound layer 11, and the conductive layer 10 is formed on the metal layer. In this case, it is preferable that the metal layer is not formed in a portion in which a three-dimensional curved shape is to be formed in a later-described step (b).

There is no particular limitation on the method for forming the metal layer, and examples thereof include vapor deposition, sputtering, and direct application (e.g., direct application of silver paste).

[Step (b)]

The step (b) is a step of allowing the film to form a three-dimensional curved shape in a self-organized manner, using as a driving force, a strain gradient in the thickness direction of the film.

A strain gradient in the thickness direction of the film can be formed by putting the conductive layer 10 and the polymer compound layer 11 respectively having predetermined thicknesses into close contact and connecting them to each other. In the example in FIG. 2, the conductive layer 10 and the polymer compound layer 11 are formed on the sacrificial layer 13, so that a strain gradient is formed in the thickness direction of the electrode film 302. By melting the sacrificial layer 13 at this time (FIG. 2(i)), the electrode film 302 is allowed to form a three-dimensional curved shape in a self-organized manner, using the strain gradient as a driving force. (FIG. 2(j)).

The sacrificial layer 13 can be melted as appropriate according to the material of the sacrificial layer 13. For example, if the sacrificial layer 13 is made of a calcium alginate gel, it is possible to melt the sacrificial layer 13 by adding a chelating agent such as sodium citrate or ethylenediaminetetraacetic acid (EDTA), an enzyme called arginase, or the like. Furthermore, if the sacrificial layer 13 is a thin metal film that can be melted by an etchant, it can be melted by an etchant, if it is poly(N-isopropylacrylamide) whose gel-sol transition can be induced by a change in the temperature, it can be melted by a change in the temperature, and, if it is a photoresist whose gel-sol transition can be induced by irradiation with ultraviolet light, it can be melted by irradiation with ultraviolet light.

[Step (c)]

Preferably, the producing method according to this embodiment includes a step (c) of allowing cells to be present on or above a surface of the layered member, after the step (a) and before the step (b).

FIG. 2(h) is a schematic view showing a state in which the cells 2 are allowed to be present on or above the surface of the electrode film 302.

The cells 2 may be present at any vertical position on or above the surface of the electrode film 302, and may be floating above the surface of the electrode film 302, or may be bonded to the surface of the electrode film 302.

In order to enclose the cells 2 in the internal space of the electrode 300 that is folded in a self-organized manner by the step (b), the distance from the surface of the electrode film 302 to each cell 2 is preferably smaller than ½ of the length of the electrode film 302 in the minor axis direction. The cells 2 are the same as those described in the section “<Electrode>” above.

There is no particular limitation on the method for allowing the cells 2 to be present on or above the surface of the electrode film 302, and it is possible to use any method. Examples of the method for allowing the cells 2 to be present on or above the surface of the electrode film 302 include a method in which a culture fluid or a suspension of the cells 2 is applied dropwise to the surface of the electrode film 302, and a method in which the electrode film 302 is immersed in a culture fluid or a suspension of the cells 2.

The number of cells 2 that are allowed to be present on or above the surface of the electrode film 302 can be controlled with the concentration of the cells 2 in the culture fluid or suspension of the cells 2.

In the above-described manner, the electrode 300 can be produced.

In an embodiment, the producing method according to this embodiment may include a step of forming a sacrificial layer on a substrate, a step of forming, on the sacrificial layer, a film including a conductive layer and a polymer compound layer 11 (layered member), a step of allowing cells 2 to be present on or above a surface of the film (layered member), and a step of melting the sacrificial layer, and allowing the film (layered member) to form a three-dimensional curved shape in a self-organized manner, using, as a driving force, a strain gradient in the thickness direction of the film (layered member). The step of forming the film (layered member) may be a step of forming a polymer compound layer on a sacrificial layer, and forming a conductive layer on the polymer compound layer; a step of forming a conductive layer on a sacrificial layer, and forming a polymer compound layer on the conductive layer; or a step of forming a first conductive layer on a sacrificial layer, forming a polymer compound layer on the conductive layer, and forming a second conductive layer on the polymer compound layer.

[Other Steps]

The producing method according to this embodiment may include other steps in addition to the steps (a) to (c). Examples of the other steps include, but are not particularly limited to, a step of patterning the electrode film 302 (patterning step) and a step of culturing cells (culturing step).

(Patterning Step)

Preferably, the method of this embodiment includes a patterning step. Preferably, the patterning step is performed after the step (a) and before the step (b). There is no particular limitation on the method for patterning the electrode film 302, and it is possible to use known patterning methods. As the patterning method, it is possible to use, for example, microfabrication techniques such as photolithography, electron beam lithography, and dry etching.

FIGS. 2(e) to 2(f) are views illustrating an example of the step of patterning the electrode film 302, and the electrode film 302 is patterned using a resist layer 15. FIG. 2(e) shows a state in which the resist layer 15 is formed on the electrode film 302. There is no particular limitation on the method for forming the resist layer 15, and it is possible to use known methods such as spin coating. A resist pattern with any shape can be obtained by exposing the resist layer 15 to light through a photomask with any shape, and developing the resist layer 15 using a developer. By etching the electrode film 302 and the sacrificial layer 13 using the resist pattern as a physical mask, the electrode film 302 patterned in any shape can be obtained (FIG. 2(f)). In the above-described patterning, the sacrificial layer 13 may or may not be patterned together with the electrode film 302, but is preferably patterned with the electrode film 302, from the viewpoints of the ease of patterning, the degradability of the sacrificial layer 13, and the like.

In the case where a fine structure is patterned, the electrode film 302 preferably has a two-dimensional plane shape, and it is possible to allow the electrode film 302 to maintain the two-dimensional plane shape, by forming the electrode film 302 on the substrate 14 and the sacrificial layer 13.

There is no particular limitation on the pattern shape formed through patterning, it is preferable to form apertures in the electrode film 302 through patterning. Accordingly, the step of patterning the electrode film 302 may be a step of forming apertures in the electrode film 302. Examples of the shape, the size, and the arrangement of the apertures are the same as those illustrated in the section of “<Electrode>” above.

Alternatively, through patterning, the electrode film 302 may be provided with any two-dimensional shape and any size. Accordingly, the step of patterning the electrode film 302 may be a step of patterning the electrode film 302 to have any two-dimensional shape and any size. For example, in the case where a tubular electrode 300 is to be obtained, the electrode film 302 is preferably patterned into a rectangular shape. The size of the rectangular shape may be selected as appropriate according to the size of cells to be enclosed, the purpose of use of the electrode 300, and the like, and may be, for example, 400 to 4000 μm long by 20 to 400 μm wide.

In the case where the formation of apertures and the formation of a two-dimensional shape are performed in the patterning of the electrode film 302, the aperture formation and the two-dimensional shape formation may be performed simultaneously, or may be performed separately through a plurality of times of patterning.

(Culturing Step)

The method of this embodiment may include a culturing step. The culturing step may be performed after the step (c) and before the step (b), or may be performed after the step (b). If the cells are adhesive cells, the cells can be bonded to the electrode film 302 by performing the culturing step after the step (c) and before the step (b). Accordingly, when allowing the electrode film 302 to form a three-dimensional curved shape in the step (b), it is possible to reliably enclose the cells in the internal space of the three-dimensional curved shape.

Furthermore, it is possible to proliferate the cells along the three-dimensional curved shape of the electrode 300 by performing the culturing step after the step (b).

As the culture conditions, it is possible to use conditions that are commonly used for culturing cells of interest, depending on the cell type.

With the method of this embodiment, along with the formation of a three-dimensional curved shape, cells are enclosed in the internal space of the three-dimensional curved shape, and it is thus possible to enclose cells in the internal space of the electrode having any three-dimensional shape. The conventional metal electrodes have a problem in that it is difficult to attach a multi-layered thin film including a metal layer along with the shape of a shape of a fine three three-dimensional living tissue, and the electrode performance is significantly reduced by partial peeling of the thin film. However, with the method of this embodiment, it is possible to allow the layered member, including the conductive layer and the polymer compound layer respectively having predetermined thicknesses, to form a three-dimensional curved shape in a self-organized manner, and, concurrently therewith, enclose cells in the internal space, and it is thus possible to form an electrode having the shape of any three-dimensional living tissue. In particular, it is possible to suppress coming off, slippage, breakage, and the like caused by a structural change into a three-dimensional shape, by using materials having high adhesion to each other as the conductive layer and the polymer compound layer. Furthermore, it is possible to obtain an electrode with any three-dimensional shape and any size by designing the shape of the electrode film into any shape and any size.

Furthermore, it is possible to maintain the electrode film in a plane state by forming the electrode film on the substrate and the sacrificial layer, so that it is possible to separate the electrode film from the substrate at any timing, and start the formation of a three-dimensional curved shape.

<Layered Member>

The layered member according to an embodiment of the present invention includes: a step of (c) of allowing cells to be present on or above a surface of the film, after the step (a) and before the step (b).

Specific examples of the layered member according to this embodiment include the layered member 303 shown in FIG. 2(g).

The substrate 14, the sacrificial layer 13, the conductive layer 10, and the polymer compound layer 11 are the same as those described in the section “<Electrode Producing Method>” above. Preferably, the layered member 303 of this embodiment includes apertures extending through the conductive layer 10 and the polymer compound layer 11. Examples of the apertures are the same as those illustrated in the section “<Electrode>” above.

The layered member according to this embodiment can be used for production of the electrode according to the above-described embodiment.

In the description above, embodiments of the present invention have been described in detail with reference to the drawings, but specific configuration thereof is not limited to those of the embodiments, and the invention encompasses other designs and the like within the range not departing the gist thereof.

### EXAMPLES

Hereinafter, the present invention will be described in more detail by way of specific examples. Note that the present invention is not limited to the examples described below.

### Example 1

### Production Example of Electrode Film

A layered member 303 was produced according to the process in FIGS. 2(a) to 2(g).

As the substrate 14, a glass substrate was used. A sodium alginate solution was spin-coated on the glass substrate, and then the resultant was immersed in 100 mM of calcium chloride solution, so that a sacrificial layer made of calcium alginate gel was formed. The thickness of the sacrificial layer can be controlled by changing the concentration of the sodium alginate solution and the speed of the spin coating. In this example, a 40-nm gel layer was formed through spin coating using 2 wt % of sodium alginate solution at 3000 rpm.

Next, the conductive layer 10 was transferred to the surface of the sacrificial layer 13. In this example, as the conductive layer 10, single-layered graphene produced through CVD on the surface of a copper foil was used. The copper foil was melted with a ferric chloride solution, and, after washing was repeated on the water surface, the single-layered graphene (the conductive layer 10) was transferred to the surface of the sacrificial layer 13. By repeating this operation, it is also possible to stack multi-layered graphene on the surface of the sacrificial layer 13.

Next, a paraxylene dimer was caused to grow through CVD, so that a polymer compound layer 11 made of polyparaxylene (Parylene) was formed on the conductive layer 10. The thickness of the polymer compound layer 11 can be controlled with the weight of paraxylene dimer that was used as a raw material of CVD growth.

In this example, 50 mg of paraxylene dimer was caused to grow through CVD on the conductive layer 10, so that a 50-nm polymer compound layer 11 was formed.

Next, a photoresist was spin-coated on the polymer compound layer 11, so that a resist layer 15 was formed. The resist layer 15 was irradiated with ultraviolet light through a photomask with a given shape, so that a physical mask with the given shape was patterned thereon. Thereafter, the polymer compound layer 11, the conductive layer 10, and the sacrificial layer 13 were etched with oxygen plasma. The etching was performed until the sacrificial layer 13 formed on the substrate 14 was reached. Lastly, the resist layer 15 was removed with acetone to expose the polymer compound layer 11 as the upper surface, thus obtaining an electrode film 302.

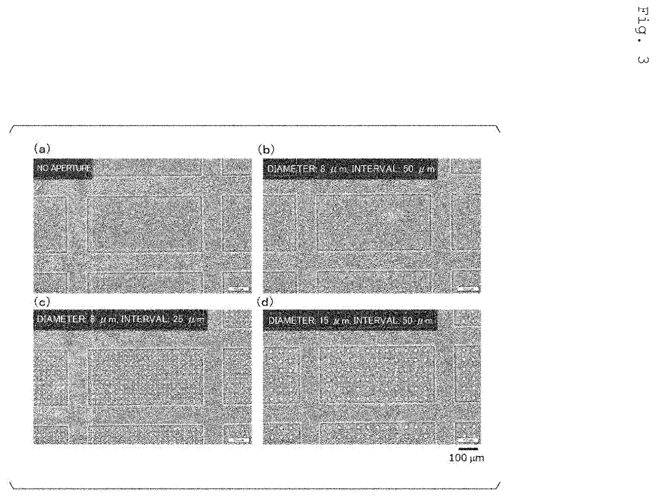

FIG. 3 shows phase-contrast microscope images of electrode films after patterning. The two-dimensional electrodes shown in FIGS. 3(a) to 3(d) are patterned into a rectangular shape of 600 μm long by 300 μm wide. FIG. 3(a) shows the film having no aperture, FIG. 3(b) shows the film having apertures with a diameter of 8 μm formed at intervals of 50 μm, FIG. 3(c) shows the film having apertures with a diameter of 8 μm formed at intervals of 25 μm, and FIG. 3(d) shows the film having apertures with a diameter of 15 μm formed at intervals of 50 μm. Note that the interval between the apertures means the center-to-center distance between adjacent apertures.

### Example 2

### Production of Electrode

An electrode was produced according to the process in FIGS. 2(h) to 2(j).

A cell culture fluid of primary culture nerve cells isolated from hippocampus tissue of a rat was seeded onto the electrode film 302, so that the nerve cells were allowed to be present on the surface of the electrode film 302.

The sacrificial layer 13 was melted by adding an EDTA solution serving as a chelating agent to the layered member 303 including the substrate 14, the sacrificial layer 13, and the electrode film 302. It was observed that bending of the electrode film 302 in the axial direction was induced after addition of the EDTA solution. Accordingly, a tubular electrode in a state in which its length in the major axis direction was maintained was obtained. The time from when the EDTA solution is added to when a tubular electrode is completed can be controlled with the final concentration of the EDTA solution that is added and the type of solution in which the substrate is immersed.

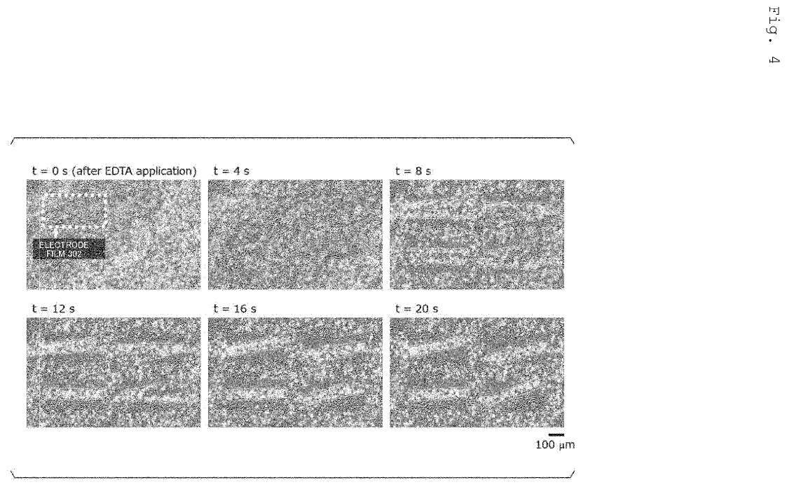

FIG. 4 shows phase-contrast microscope images showing an enclosing process of the primary culture nerve cells with self-organized curving of the electrode film 302. FIG. 4 shows phase-contrast microscope images obtained 0 seconds (t=0 s), 4 seconds (t=4 s), 8 seconds (t=8 s), 12 seconds (t=12 s), 16 seconds (t=16 s), or 20 seconds (t=20 s) after addition of EDTA. It was possible to observe the process in which, with the curving of the electrode film, the cells floating in the culture fluid were enclosed into the internal space of the three-dimensional curved shape formed by the electrode film. It was possible to confirm that the electrode film 302 including the conductive layer 10 and the polymer compound layer 11 was capable of being curved in a self-organized manner, and that the cells were enclosed in the process of the curving.

### Example 3

### Induction of Neurites in Cell-Enclosing Electrode

The cell-enclosing electrode produced in the above-described manner was cultured on a culture dish, and whether or not the cells enclosed by the electrode were able to extend neurites to the external environment was determined.

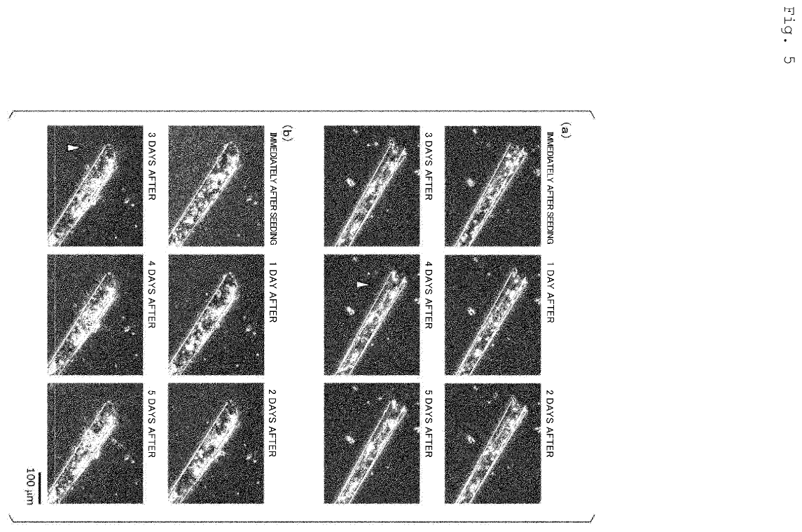

FIG. 5 shows phase-contrast microscope images obtained by performing time-lapse imaging on the cells enclosed by the cell-enclosing electrode. FIG. 5(a) shows the electrode having no aperture on the electrode surface, and FIG. 5(b) shows the electrode having apertures with a diameter of 8 μm on the electrode surface.

Regardless of the presence or absence of apertures, the cells enclosed by the electrode were maintained during the five-day observation period after culture. In the electrode having no aperture, the extension of neurites from the tubular end of the tubular electrode with the passage of the culture days was observed, but no extension of neurites from the tubular wall surface was observed (FIG. 5(a)). On the other hand, in the electrode in which apertures with a diameter of 8 μm were formed, neurites extended from the apertures, so that the extension of neurites over a wide range on the culture dish was observed.

This example shows a case where both ends of the electrode are open. On the other hand, one end or both ends of the electrode may be closed. When one end of the electrode is closed, it is possible to limit the movement of cells into and out of the electrode to movement from the open end. When both ends of the electrode are closed, it is possible to inhibit the movement of cells into and out of the outside of the electrode.

### Example 4

### Change between the Electrical Properties Before and After Self-Folding

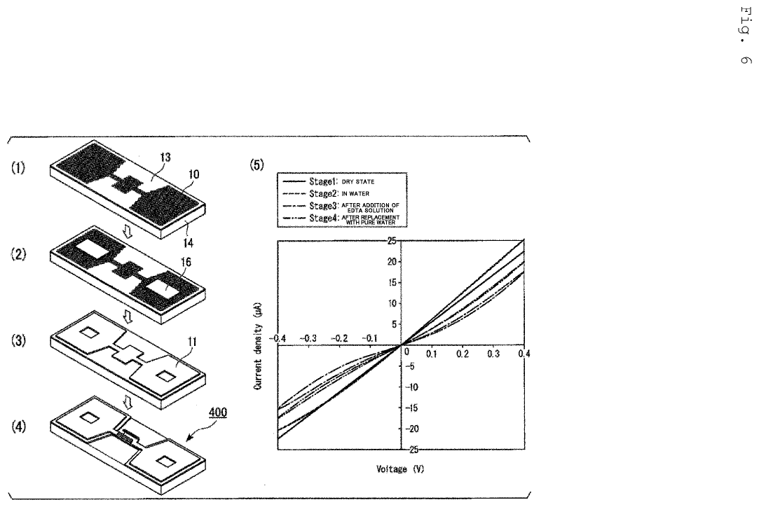

An electrode with a three-dimensional curved shape was produced according to the process shown in FIGS. 6(1) to 6(4).

A sacrificial layer 13 was formed on a substrate 14, and a conductive layer 10 was transferred to the surface thereof (FIG. 6(1)). The substrate 14, the sacrificial layer 13, and the conductive layer 10 were as in Example 1. Next, a gold electrode (a metal layer 16) was vapor-deposited to each of the two ends of the conductive layer 10 (FIG. 6(2)). Subsequently, a Parylene layer (the polymer compound layer 11) was vapor-deposited, and the surface thereof was subjected to patterning (FIG. 6(3)). Then, an EDTA solution was added, and thus an electrode 400 whose structure was changed into a recessed shape was obtained (FIG. 6(4)).

FIG. 6(5) shows I-V curves of the electrode 400 before and after self-folding described above.

It was seen from the result shown in FIG. 6(1) that a change in the resistance caused by a change in the structure was observed, but breakage did not occur even when the three-dimensional curved shape was formed, and stable conductivity was maintained.

### INDUSTRIAL APPLICABILITY

According to the present invention, it is possible to provide an electrode that can enclose cells and be implanted in a living body, a method for producing the same, and a layered member for use in production of the electrode. The above-described electrode can be used as transplant tissue by enclosing cells. In a cell-enclosing electrode that has been transplanted in a living body, the contact between transplant tissue and the electrode is secured, and it is therefore possible to measure the activity of the transplant tissue in the living body over a long period of time.

### REFERENCE SIGNS LIST

2 Cell
3 Host tissue
10, 10a, 10b Conductive layer
10 Polymer compound layer
11 Aperture
13 Sacrificial layer
14 Substrate
15 Resist layer
16 Metal layer
20 Cell body
21 Neurite
30 Nerve cell of host tissue
31 Cell body of nerve cell of host tissue
32 Neurite of nerve cell of host tissue
100, 200, 300 Electrode
101, 201, 302 Electrode film
303 Layered member

1. An electrode having an internal space, the electrode comprising a film including a layer containing a conductive material (conductive layer), wherein the internal space is formed by the film being curved.

2. The electrode according to claim 1, wherein cells are present in the internal space.

3. The electrode according to claim 1,
wherein the film has apertures that communicate the internal space with an external space of the electrode.

4. The electrode according to any one of claims 1, wherein the electrode has a tubular shape.

5. The electrode according to claim 4, wherein one end or both ends of the tubular shape are closed.

6. The electrode according to claim 1, wherein the film further comprises a layer containing a polymer compound (polymer compound layer).

7. The electrode according to claim 6, wherein the polymer compound layer and the conductive layer are made of a material having light transmittance.

8. The electrode according to claim 1, wherein the conductive material is a conductive carbon material.

9. A method for producing an electrode, comprising: a step (a) of forming a film including a layer containing a polymer compound (polymer compound layer), and a layer containing a conductive material (conductive layer); and a step (b) of allowing the film to form a three-dimensional curved shape in a self-organized manner, using, as a driving force, a strain gradient in a thickness direction of the film.

10. The method for producing an electrode according to claim 9, further comprising a step of (c) of allowing cells to be present on or above a surface of the film, after the step (a) and before the step (b).

11. A layered member comprising: a substrate; a sacrificial layer stacked on the substrate; a layer containing a conductive material (conductive layer), the layer being stacked on the sacrificial layer; and a layer containing a polymer compound (polymer compound layer), the layer being stacked on the conductive layer.
